SEQUENCE LISTING (14 sequences)

SEQ ID NO: 1 - DNA, 24 bp, Artificial
  Primer - bsLTRrv1
cccaaaataaacaatcaatcaatc

SEQ ID NO: 2 - DNA, 23 bp, Artificial
  Primer - bsLTRfw2
gagaatagggaagtttagattaa

SEQ ID NO: 3 - DNA, 31 bp, Artificial
  Probe - bsLTR M1
aaacgcgcgaacaaaaacgaaaaacgaacta

SEQ ID NO: 4 - DNA, 34 bp, Artificial
  Probe - bsLTR UM2
aaaccatatctaaaaaccatctattcttaccccc

SEQ ID NO: 5 - DNA, 28 bp, Artificial
  Primer - bsLTRtqm Fw2
ggttaaataggatatttgtggtgagtag

SEQ ID NO: 6 - DNA, 27 bp, Artificial
  Primer - bsLTRtqm Rv1
aattcttaaacctcttttataaaactc

SEQ ID NO: 7 - DNA, 21 bp, Artificial
  Primer - H19 ICR1 fw
acattcacacgagcatccagg

SEQ ID NO: 8 - DNA, 21 bp, Artificial
  Primer - H19 ICR1 rv
gctctttaggtttggcgcaat

SEQ ID NO: 9 - DNA, 21 bp, Artificial
  Primer - L2N (Msp1)
atctgtggtgagcagtttcgg

SEQ ID NO: 10 - DNA, 21 bp, Artificial
  Primer - L3N
agaggctttattaggaacggg

SEQ ID NO: 11 - DNA, 21 bp, Artificial
  Primer - L5
caacctggaaacatctgatgg

SEQ ID NO: 12 - DNA, 20 bp, Artificial
  Primer - L6
cccaagaacccttactcggc

SEQ ID NO: 13 - DNA, 20 bp, Artificial
  Primer - L5N
cttgaaactgctgagggtta

SEQ ID NO: 14 - DNA, 21 bp, Artificial
  Primer - L6N
agtcctccgatagactgtgtc

(19) United States
(12) Patent Application Publication
(10) Pub. No.: US 2010/0003677 A1
(43) Pub. Date: Jan. 7, 2010

(54) IDENTIFICATION OF TUMOR SUPPRESSOR GENES IN AN ACUTE MYELOID LEUKAEMIA MODEL

(75) Inventors: Ivo Paul Touw, Rotterdam (NL); Stefanus Joseph Erkeland, Rotterdam (NL); Renee Beekman, Rotterdam (NL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: ERASMUS UNIVERSITY MEDICAL CENTER ROTTERDAM, Rotterdam (NL)

(21) Appl. No.: 12/303,067

(22) PCT Filed: Jun. 1, 2006
(86) PCT No.: PCT/NL2006/000273

§ 371 Date: May 4, 2009

(51) Int. Cl. C12Q 1/68; C07H 21/04

(52) U.S. Cl. 435/6; 536/23.5

(57) ABSTRACT

The present invention comprises a method method to identify tumor suppressor genes by detecting genes in a mouse retroviral insertion mutagenesis model which expression is inhibited by methylation of the viral insertion or the VIS-flanking gene. This is preferably accomplished by first randomly cutting the mouse genomic DNA, immunoprecipitating the methylated DNA and amplifying the VIS-flanking DNA by inverse PCR, optionally followed by cloning and sequencing of the amplicons.

Next to the already known tumor suppressor genes Smad1 and Mad1-like, several putative tumor suppressor genes have been found. The tumor suppressing properties of these genes, as indicated in Table 3 also form part of the present invention. Further use of these genes and/or its substrates or downstream products, for diagnosis and therapy of cancer, preferably AML is envisaged.

The invention is related to the field of cancer, more specifically to the field of leukaemia and to the detection of genes playing a role in the development of said cancer.

Retroviral integration mutagenesis is considered a powerful tool to identify cancer genes in mice (Suzuki, T., et al, 2002, Nat. Genet. 32:166-174; Erkeland, S. J. et al., 2004, J. Virol. 78:1971-1980; Joosten, M. et al., 2002, Oncogene 21:7247-7255; Mikkers, H. et al., 200, Nat. Genet. 32:153-159; Neil, J. C. and Cameron, E. R., 2002, Cancer Cell 2:253-255; Akagi, K. et al., 2004, Nucleic Acids Res. 32: D523-527). Identification of genes generally takes place by amplification of the genomic sequences flanking the virus integration site (VIS), whereby VIS-flanking genes common to independent tumors (i.e. common VIS genes) are considered bona fide disease genes. However, VIS genes not yet found common often also belong to gene classes associated with cancer and may qualify as disease genes. Further, genes located more distantly from the VIS may also be involved in disease, but the likelihood of this happening and the influence of the distance between the gene and the VIS is unknown. Recently, it has been established that the genes, detected in this mouse model, have clinical relevance for human cancers (Erkeland, S. J. et al., 2006, Cancer Res. 66:622-626).

It is generally assumed that expression of VIS flanking genes is most frequently increased due to the transcription enhancing activities of the viral LTR. Thus, in that case it would only be possible to find genes that play an active role in the forming or maintenance of the tumor. It would be desirable to search for (common) VIS-flanking genes, that are effective in the above indicated mouse retroviral integration mutagenesis models, of which the expression is decreased by the viral insertion, since these genes would likely act normally as tumor suppressor genes. With the current models, it is very difficult to discriminate between genes that are overexpressed and genes of which the expression is inhibited.

Thus, there is need for a method using retroviral integration mutagenesis, which allows for the detection of genes inhibited because of the viral insertion.

The inventors now have discovered that such genes can be identified by investigating the methylation pattern which in some instances occurs during retroviral integration. As is well known, one of the defence mechanisms of cells against viral attack is methylation of the viral DNA, thereby marking said DNA as ‘foreign’, whereafter the methylated DNA is silenced by endogenous silencing mechanisms. The methylation takes place at the so-called CpG islands in the LTR of the virus, through mechanisms which are well known in the art. In this way expression of the viral DNA and the DNA of the VIS-flanking genes is prohibited. It has further appeared that this methylation is able to spread over the VIS-flanking genes, which thus results in further inactivation (inhibition of expression) of the VIS-flanking genes.

One embodiment of the present invention is a method to identify tumor suppressor genes by detecting genes in a mouse retroviral insertion mutagenesis model which expression is inhibited by methylation of the viral insertion or the VIS-flanking gene. This is preferably accomplished by first randomly cutting the mouse genomic DNA, immunoprecipitating the methylated DNA and amplifying the VIS-flanking DNA by inverse PCR, optionally followed by cloning and sequencing of the amplicons.

Next to the already known tumor suppressor genes Smad1 and Mad1-like, several putative tumor suppressor genes have been found. The tumor suppressing properties of these genes, as indicated in Table 3 also form part of the present invention.

### LEGENDS TO THE FIGURES

FIG. 1. Taqman strategy for detection of methylated CpG in integrated LTR's of MuLV. These LTR's are known to possess 516 CpG's. Analysis is focused on CpG's 161-337, which are core CpG's known to be target for methylation. Two rounds of PCR are performed on bisulphite-treated genomic DNA. The first regular PCR is done with methylation insensitive primers to amplify the region containing CpG's 161-337. The second (Taqman PCR) round is performed with nested primers within this region in which the reverse (RV) primer is either methylation sensitive (M1) or methylation insensitive (M1u). Signals are quantified by Taqman light cycler. Probe and primer compositions are given in text. Delta Ct values calculated by subtracting Ct values obtained with RV primer M1u from Ct values obtained with RV primer M1 provide a quantitative measure of the methylation status of LTRs in a given tumor sample.

FIG. 2. Results of methylation detection experiments (Taqman) in leukaemia samples from mice infected with the Graffi 1.4 murine leukaemia virus. To generate a reference line for the Taqman assay, mixing experiments with methylated LTR-containing plasmid (341) and nonmethylated LTR-containing plasmid (340) were performed and delta Ct values calculated as described with FIG. 1 (upper Table). These cloned LTR sequences are derived from bisulphite-treated genomic DNA from a Graffi-1.4-induced tumor. PCR-amplified LTR sequences from this tumor were cloned into TA vector and sequenced to detect methylation status. This showed that the assay is linear between delta Ct values 0 and 8.00 (Graph). Based on these values, 5 categories of methylation, (<5; 5-12.5; 12.5-25; 25-50; and 50-100) were defined (lower Table).

FIG. 3. Results of the agarose gel with the amplicons from the inverse PCR after MeDIP enrichment for methylated DNA. Tumor cell samples from different leukemic mice (99-12, 99-49, etc), derived from liver (Li) spleen (Spl) or bone marrow (BM) were analyzed. Bands with sizes greater than the viral LTR sequence only (marked by line) represent fragments that consist in part of LTR sequence and in part of flanking genomic sequences.

### DETAILED DESCRIPTION OF THE INVENTION

In the research that led to the present invention, a number of genomic regions were identified to be involved in tumor development by proviral tagging. Proviral tagging (Berns. 1988. Arch Viro. 1.102:1-18; Kim et al. 2003. J Virol. 77:2056-62) is a method that uses a retrovirus to infect normal vertebrate cells. After infection, the virus integrates into the genome thereby disrupting the local organization of the genome. This integration affects the expression or function of genes, depending on the integration site of the virus, which may for instance be in a coding region, a regulatory region or a region nearby a gene. If a cellular gene involved in tumor development is affected, the cell will acquire a selective advantage to develop into a tumor as compared to cells in which no genes involved in tumor development are affected. As a result, all cells within the tumor originating from the cell affected in a gene involved in tumor development will carry the same proviral integration. Through analysis of the region nearby the retroviral integration site, the affected gene can be identified.

Mouse retroviral insertion mutagenesis models are known for several types of cancer. For acute myeloid leukaemia (AML) the Graffi 1.4 (Gr-1.4), BXH2 and AKxD murine leukaemia virus (MuLV) models have been proven useful for finding genes involved in the development, maintenance and spread of leukaemia.

Acute myeloid leukemia (AML) is the most frequent form of acute leukemia in adults and is one of the most aggressive forms of leukemia, which is acutely life threatening unless treated with different kinds of chemotherapy. Depending on the AML subtype determined by various clinical parameters, including age, and laboratory findings, for instance cytogenetic features, allogeneic stem cell transplantation might follow the remission induction by chemotherapy. The 5 years overall and disease free survival rate of adult AML is currently in the order of 35-40%. There is a strong need for a more precise diagnosis of AML, which allows for better distinction between the prognostic subtypes and for new therapeutic strategies for the large contingent of patients that can not be cured to date. The currently available laboratory techniques allow for a prognostic classification, but this is still far from optimal. Still, most patients cannot satisfactorily be risk-stratified and still a majority of patients are not cured by currently available treatment modalities.

The pathogenesis of leukemia is complex. Before becoming clinically overt, leukemic cells have acquired multiple defects in regulatory genes that control normal blood cell production. In human leukemia, until now only few of these genes have been identified, mainly by virtue of the fact that these genes were located in critical chromosomal regions involved in specific chromosome translocations found in human AML. Studies in mice, particularly those involving retroviral tagging, have yielded only relatively small numbers of retroviral insertions and target genes per study, but have nonetheless made clear that there are at least a few hundred genes that can be involved in the pathogenesis of murine leukemia. There is a strong conservation between the mouse and human hematopoietic systems, as is for instance evident from the fact that the biological properties of the hematopoietic progenitor cells and the regulators (hematopoietic growth factors) are largely similar. Therefore, it is not surprising, that is recently has been established (Erkeland, S. J. et al., 2006) that these genes have human, clinical relevance.

Also for other cancers such models exist, e.g. mice infected with murine mammalian tumor virus (MMTV) as a model for breast cancer and mice infected with e.g., Moloney virus or Cas-Br-M virus for B and T cell lymphoma's.

Because MuLV preferentially, albeit not exclusively, integrate into the 5′ promoter region of genes, it is generally assumed that expression of VIS-flanking genes is most frequently increased due to the transcription enhancing activities of the viral LTR. However, CpG islands in the viral LTR are a potential target for de novo methylation, which could form the initiating event to silencing the (expression of the) viral insert and the VIS-flanking genes.

In mammalian cells, approximately 3.5 to 5% of the cytosine residues in genomic DNA are present as 5-methylcytosine (Ehrlich et al., 1982, Nucl. Acids Res. 10:2709-2721). This modification of cytosine takes place after DNA replication and is catalyzed by DNA methyltransferase using S-adenosyl-methionine as the methyl donor. Approximately 70% to 80% of 5-methylcytosine residues are found in the CpG sequence (Bird, 1986, Nature 321:209-213). This sequence, when found at high frequency in the genome, is referred to as CpG islands. Unmethylated CpG islands are associated with housekeeping genes, while the islands of many tissue-specific genes are methylated, except in the tissue where they are expressed (Yevin and Razin, 1993, in DNA Methylation: Molecular Biology and Biological Significance. Birkhauer Verlag, Basel, p. 523-568). This methylation of DNA has been proposed to play an important role in the control of expression of different genes in eukaryotic cells during embryonic development. Consistent with this hypothesis, inhibition of DNA methylation has been found to induce differentiation in mammalian cells (Jones and Taylor, 1980, Cell 20:85-93).

Methylation of DNA in the regulatory region of a gene can inhibit transcription of the gene. This is probably caused by intrusion of the 5-methylcytosine into the major groove of the DNA helix, which interferes with the binding of transcription factors.

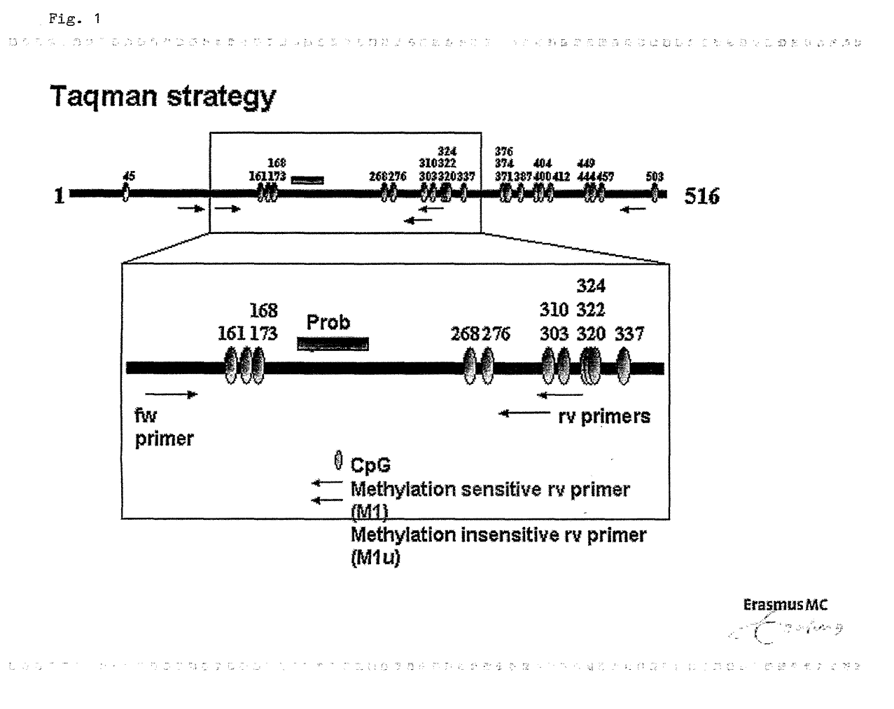

Existence of methylation has been shown in the present mouse model by a methylation sensitive Q-PCR (FIG. 1). However, other strategies for demonstrating methylation, such as MeDIP and methylation sensitive restriction enzyme digestion, may be employed. By Q-PCR, it was found that LTR methylation in the applied model occurs with variable frequencies, ranging from <5% to 50-100% (See Table 2). However, these estimations are currently provisional and need to be verified by other methods.

Since tumors developed in these cases, where the proviral insertion (and possibly a part of the flanking genes) were methylated and thus the expression of these genes was inhibited, this means that knock-out of these genes apparently is a trigger for the development or maintenance of the tumor. Thus, it is envisaged, that these genes, which are subject to transcription and translation in a normal, wild-type cell, would then act as tumor suppressors.

As is exemplified in the Experimental part, it is possible to retrieve the identity of the VIS-flanking genes from samples of the tumors. In the present invention, this is accomplished by digesting the genomic DNA with a restriction enzyme, enrichment of methylated DNA fragments by immunoprecipitation and applying an inverse PCR on these fragments. The amplified fragments are then subjected to gel electrophoresis, which yields several bands, which can be sequenced and from which the identity of the genes can be retrieved.

However, the invention is not limited to the above-applied method. Any method known in the art which enables isolation of VIS-flanking genes surrounding a methylated viral insert would be feasible to detect potential tumor suppressor genes.

There are several ways whereby the identified genes can be assayed for their tumor suppressor function. Firstly, growth factor dependent cell lines are available that faithfully recapitulate normal myeloid cell proliferation, survival and differentiation in response to exogenous stimuli, such as granulocyte colony-stimulating factor (G-CSF). Based on the cellular features of AML cells, it is a reasonable assumption that reduced expression of tumor suppressor genes in this model will have negative effects on the induction of myeloid differentiation and stress-induced (e.g., by growth factor deprivation) apoptosis induction, or positive effects on pro-survival and proliferation signaling pathways. A murine interleukin3-dependent cell-line engineered to express the human G-CSF receptor is particularly suitable for these studies (De Koning et al, Blood 91: 1924, 1998). Genes of interest (single or multiple) can be knocked-down in these cells using siRNA or shRNA approaches and changes in cell proliferation, survival and differentiation and expression of genes and activation of signaling pathways involved herein can be taken as functional endpoints. This analysis can be extended to primary bone marrow stem cells and progenitor cells using in vitro and in vivo approaches in mice. For the latter, hematopoietic stem cells transduced with siRNA or shRNA can be transplanted into irradiated recipient mice, which can be monitored for defects in blood cell production and possible development of leukemia. These experiments may also be performed in (genetically modified) mouse strains that are already predisposed to tumor development due to other genetic abnormalities. In addition, genetic approaches may be taken to knock out genes in mouse embryonic stem cells to generate gene deficient mouse strains and to cross these mice with relevant tumor-prone strains to study cooperativity of gene defects in tumor development.

Thus, an embodiment of the present invention are the tumor suppressor genes, that were found in the VIS-flanking genes of the methylated samples. These genes are listed in Table 3. The person skilled in the art will recognise that some of the genes found are already known as tumor suppressor genes (Smad1 and Mad1-like), but the largest part of the listed genes are unknown to play a role in suppression of tumors. Ideally, a tumor suppressor gene is found in more than one sample, which confirms its importance in tumor suppression. Expression of the genes of interest will be analyzed in clinical AML, by employing gene array-based expression profiling (Valk et al, N Engl. J Med 2004 Apr. 15; 350(16):1617-28), to determine their relevance for human disease and to establish their potential prognostic value, along the lines similar to those described in the study by Erkeland et al (Erkeland, S. J. et al., 2006, Cancer Res. 66:622-626).

The genes from Table 3, and optionally further identified by the above described expression profiling may be used to develop diagnostic tools to further risk-stratify cancer, in particular AML. As is shown in WO 2005/080601 genetic expression information, alongside with clinical parameters, can be used to classify AML, and, on basis of said classification, predictions can be made about responsiveness to a particular therapy. It is envisaged that the genes of the present invention will be a further aid for such a classification and determination of susceptibility to therapy.

The genes from Table 3 may potentially also form the starting point for the design of therapeutic strategies. One such a strategy can be to increase expression of the gene in vivo, e.g. by enhancing the activity of the promoter and/or by genetic therapies using (viral) vectors coding for the gene. Another strategy aimed at restoring activities of critical downstream substrates of these genes is envisaged. Now the tumor suppressor genes of the invention are known, a person skilled in the art can easily detect downstream gene products and/or substrates. Depending on the nature of such products and/or substrates therapy will consist of administration of these products and/or substrates to restore natural levels, or closing down pathways that would deplete the produced amounts by e.g. siRNA treatment.

Experimental Part

### 1. Protocols

I. PCR to Amplify LTR Sequences after Bisulphite Treatment
Take 2 ul of DNA from tumor samples and treat with bisulphite as described in protocol of DNA EZ methylation kit D5002 (ZymoResearch/BaseClear)

### Use 1 ul for PCR:

| | |
|---|---|
| 1 ul template | 5′ 94° C. |
| 1 ul bsLTRrv1 | 10 cycles |
| 1 ul bsLTRfw2 | 30″ 94 C. |
| 6 ul NTP's (1.25 mM/NTP) | 30″ 50 C. |
| 5 ul buffer | 1′ 72 C. |
| 0.25 ul Taq | 7′ 72 C. |
| storage at 4 C. | |
| bsLTRrv1: CCCAAAATAAACAATCAATCAATC | |
| bsLTRfw2: GAGAATAGGGAAGTTTAGATTAA | |

### II. Quantification of Methylated LTR by Quantitative PCR (Taqman)

2 μl DNA (from PCR I)
0.25 μl dNTP's (10 mM)
0.25 μl probe: bsLTR M1
(5′-AAACGCGCGAACAAAAACGAAAAACGAACTA-3′)
or
UM2 (5 pmol/μl)
(AAACCATATCTAAAAACCATCTATTCTTACCCCC)
2.5 μl buffer A
0.125 μl Ampli Taq Gold
1 μl bsLTRtqm Fw2 (10 pmol/μl)
(GGTTAAATAGGATATTTGTGGTGAGTAG)
1 μl bsLTRtqm Rv1 (10 pmol/μl)
(AATTCTTAAACCTCTTTTATAAAACTC)
5 μl MgCl2 (25 mM)
12.875 μl MQ (end volume 25 μl)

### Cycling Protocol:

| | |
|---|---|
| 1x | 10 min 95° C. |
| 45x | 15 sec 95° C. |
| | 30 sec 58° C. |
| | 30 sec 60° C. |

### III. MeDIP on Methylated CpGs

### Reagents

Proteinase K (10 mg/ml)
Mbo1 enzyme (GATC) & Neb 3 buffer; MboI R0147L, Biolabs
α-Methylcytidine antibody (1 μg/μl) BI-MECY-0500, Eurogentech, Maastricht
Pre-immune serum IgG (12 μl/μl, diluted to 1 μg/μl), Mouse IgG technical grade from serum, Sigma, Zwijndrecht
Ip buffer (should be cold!): PBS solution

### 0.05% Triton-X-100

100% ethanol

### 3M NaAc, ph 5.5

### Phenol/chloroform

Glycogen 20 μg/μl Roche 901393 (optional)
Protein G-Sepharose beads

### Protocol

### Day 1

### Digestion of Genomic DNA

Take 10 microgram genomic DNA and digest o/n with 50 units of Mbo1 (10 μl) in total of 100 μl (Neb buffer 3)

### Day 2

### Antibody Incubation

Take 2×40 μl of digestion product and denaturise DNA for 10′ at 95° (also for enzyme inactivation)
Keep 4 μl as 10% input control, add 200 μl IP-buffer and put on at 4° on a roller until prot K will be added
Put denaturised samples directly on ice
Add 20 μg antibody (20 μl) and add total volume up to 500 μl with IP-buffer (1 sample with α-methylcytidine and 1 with mouse pre-immune serum IgG)
Incubate samples for 2 hr at 4° on a roller
Incubation with Dynabeads

Wash 60 μl of Dynabeads (M-280 Sheep anti mouse IgG 112.01, Dynal Biotech) per tumor sample; 3×

Add 1000 μl IP-buffer to pooled beads and place in magnet for 2 minutes, remove supernatant, at the last step: resuspend beads thoroughly in 110 μl IP-buffer per tumor sample
Add 50 μl of beads to the + and − sample of each tumor and incubate for 2 hr at 4° on a roller

### Washing Beads

Wash samples 3× with 700 μl IP-buffer, finally resuspend beads in 200 μl IP-buffer
Add 200 μl IP-buffer to the 10% input sample

### Elution of DNA

Add 2 μl proteinase K (=20 μg) to the input, + and − samples and incubate or for 3 hrs at 50°
Discard beads and keep the supernatant

### DNA Recovery

Add 200 μl phenol/chloroform and spin down (spin 5′ at 13 k rpm)
Collect supernatant

### Add 500 μl 100% EtOH

Add 20 μl 3M NaAc pH 5.5 and optional 0.5 μl (10 μg) glycogen
Incubate o/n at −20° C. to precipitate DNA (or at −80° C. until sample is frozen)

### Day 3

### Spin 30′ 13 k, 4° C.

Decant supernatant
Add 500 μl ice cold 70% EtOH

### Spin 10′ 13 k, 4° C.

### Dilute DNA in 20 μl MQ

PCR after MeDIP

### Samples

### Use 1 μl of the MeDIPped DNA for PCR

PCR on 3 different samples:
Input control (should always be positive)
IgG control (controls for the amount of aspecific binding)
A-methylcytidine sample (positive if DNA was methylated)

### Sequences

Amplification of 3 different sequences
H19: positive control, H19 ICR1 fw
(ACATTCACACGAGCATCCAGG) ×
H19 ICR1 rv (GCTCTTTAGGTTTGGCGCAAT) 125 bp
LTR: L2N (Msp1) (ATCTGTGGTGAGCAGTTTCGG) ×
L3N (AGAGGCTTTATTAGGAACGGG) 287 bp

### Tm: 58°

### Elongation: 30″

Expected result:

| | | | α- |
|---|---|---|---|
| Primer | Input | IgG | methylcytidine |
| H19 | + | − | + |
| LTR | + | − | + (if methylated) |
| | | | − (if not |
| | | | methylated) |

III. Inverse PCR after MeDIP

Take 8 μl MeDIPped DNA

Add 2 μl dilution buffer, and add up to 10 μl with MQ-H₂O

Add 10μ T4 DNA ligation buffer

Add 1 μl T4 DNA ligase

Leave at RT for 15′

Heat inactivate T4 DNA ligase at 65° for 15′

Take 2 μl for PCR in total of 50 μl (L5×L6)

Take 2 μl of this dilution and perform nested PCR (L5N×L6N)

### PCR Program: INVPCR1 (60°) and INVPCR2 (56°)

10′ 94°
30 cycles
30″ 94°

### 30″ 60° (L5×L6) or 56° (L5N×L6N)

3′ 72°
End cycles
5′ 72°
4° storage

### Primers:

L5: CAACCTGGAAACATCTGATGG
L6: CCCAAGAACCCTTACTCGGC
L5N: CTTGAAACTGCTGAGGGTTA
L6N: AGTCCTCCGATAGACTGTGTC

### I. Quantification of Methylated LTR by Quantitative PCR (Taqman)

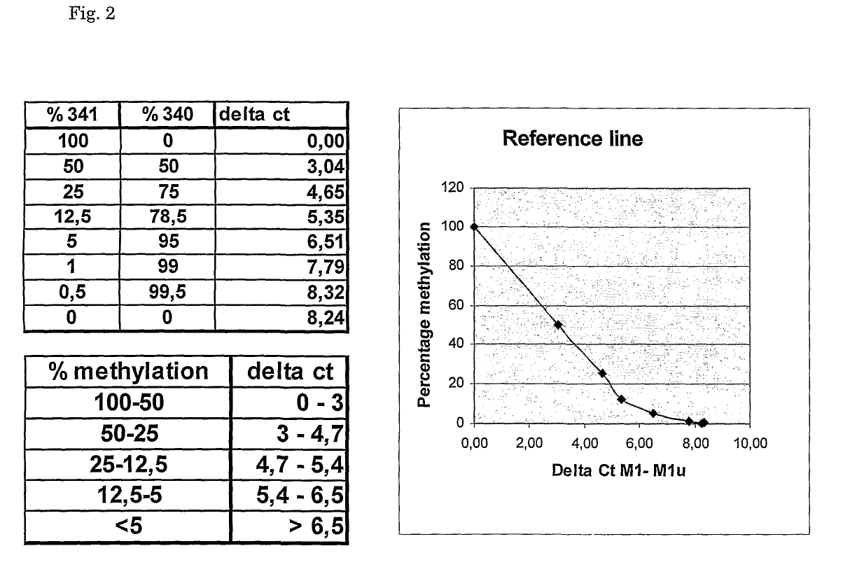

To establish whether integrated proviral sequences, specifically CpG islands in the long terminal repeat (LTR) sequences of Graffi 1.4 murine leukaemia virus (Gr-1.4 MuLV) were methylated in Gr-1.4 MuLV-induced tumors, and to what extent, a quantitative method involving methylation specific PCR, based on Taqman technology, was developed. (FIG. 1). Methylation specific PCR (MSP) is a well established technique in genome research (Derks et al, Cell Oncol. 2004; 26 (5-6):291-9). To establish linearity of this assay, an experiment was performed with plasmid DNA's containing sequences derived either from the unmethylated LTR (plasmid 340) or the methylated LTR (plasmid 341). Based on this, a reference line was generated and methylation status categories defined (FIG. 2). Next it was established that genomic DNA samples from normal somatic tissues (bone marrow, liver, spleen) do not give a specific signal in this assay, in line with the fact that these normal tissues are not expected not contain (methylated) Graffi 1.4 LTR sequences (Table 1). We then screened all Graffi 1.4-induced tumors (n=81). Distinct methylation categories were defined: high (n=7), medium-high (n=15), medium (n=12), low (n=20) and very low to none (n=27) (high and medium high samples shown in Table 2).

TABLE 1
Ct values in normal tissue samples. Ct value <30 for
M1u and <34 for M1 indicate that no methylated
LTRs are present.

| Tissue | Ct M1u | Ct M1 |
|---|---|---|
| normal bone marrow | 31.8 | 35.4 |
| Normal liver | 31.4 | 37.9 |
| Normal spleen | 31.8 | 40.8 |
| MQ (nested PCR) | 30.3 | 33.9 |
| MQ (Taqman) | Not determined | Not determined |

TABLE 2
Methylation status of high and
medium high methylated samples.

| | | | % |
|---|---|---|---|
| | | delta | methylation |
| sample | organ | Ct | mean |
| 99-12 | lymph node | 1.6 | 100-50 |
| 99-23 | bone marrow | 2.1 | 100-50 |
| 99-49 | bone marrow | 2.2 | 100-50 |
| 99-5 | liver | 2.3 | 100-50 |
| 99-20 | spleen | 2.4 | 100-50 |
| 99-10 | liver | 2.5 | 100-50 |
| 99-44 | bone marrow | 2.6 | 100-50 |
| 99-55 | spleen | 3.1 | 50-25 |
| 00-10 | liver | 3.2 | 50-25 |
| 99-29 | bone marrow | 3.4 | 50-25 |
| 99-16 | spleen | 3.5 | 50-25 |
| 99-34 | spleen | 3.7 | 50-25 |
| 99-33 | bone marrow | 3.7 | 50-25 |
| 00-14 | liver | 3.7 | 50-25 |
| 99-36 | bone marrow | 3.9 | 50-25 |
| 00-9 | liver | 4.0 | 50-25 |
| 00-17 | liver | 4.1 | 50-25 |
| 00-22 | liver | 4.2 | 50-25 |
| 99-19 | spleen | 4.3 | 50-25 |
| 99-48 | liver | 4.4 | 50-25 |
| 00-4 | spleen | 4.5 | 50-25 |
| 99-18 | spleen | 4.5 | 50-25 |

### II. MeDIP on Methylated CpGs

The genomic DNA was digested with Mbo1. The fragmented DNA was enriched for methylated DNA by immunoprecipitation with MeDIP (incubation with antibodies directed against 5-methyl-cytosine, α-5MC). Primers L2N and L3N were generated to detect methylated LTR after MeDIP. Primers were also generated for the methylation imprinted gene H19, serving as positive control on the MeDIP procedure. Enrichment of LTRs after MeDIP with α5-mC was found in 25/34 samples tested thus far. Positive signals were found in all methylation categories, with generally the highest signal in the high to medium high methylation categories and lower signals in the low to very low categories. As expected, MeDIP on normal hematopoietic tissues was negative for LTR, but positive for the methylation imprinted gene H19.

III. Inverse PCR after MeDIP and Identification of Flanking Genomic Regions

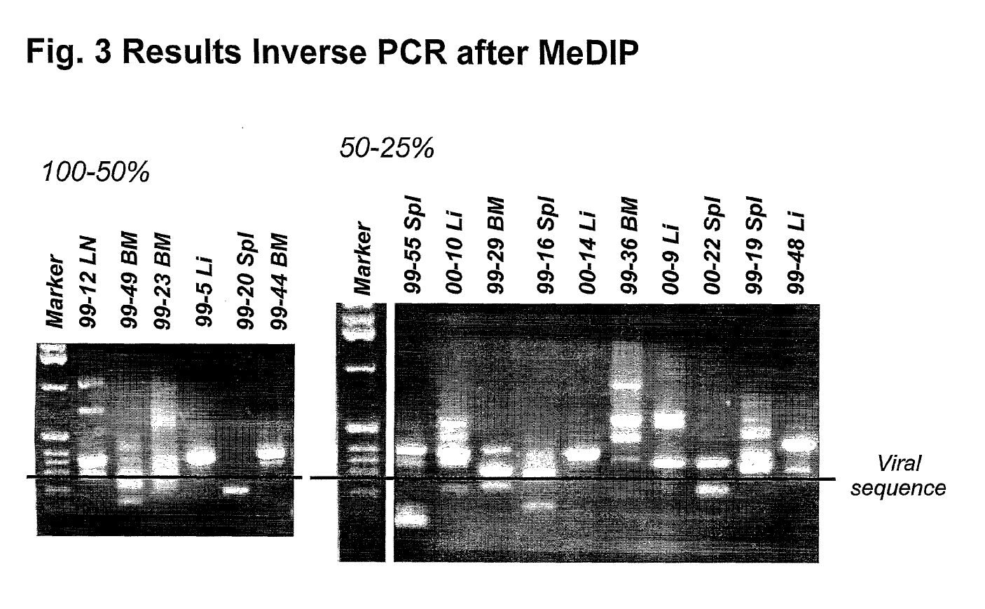

MeDIP/iPCR was performed on the positively responding samples (all high and medium high methylation samples, except 99-10, 99-33, 99-34 and 00-17, and samples 00-18 (spleen), 99-3 (liver), 99-47 (liver), 00-19 (bone marrow), 99-56 (spleen), 99-7 (liver) and 99-58 (spleen) from the middle methylation samples and samples 00-5 (spleen) and 99-45 (bone marrow from the low methylation samples). This resulted in 1 to 7 bands per tumor sample (FIG. 3, results of medium and low methylation samples not shown). Bands were isolated and subjected to nucleotide sequencing to identify flanking sequences. Genes located within a distance of 500 Kb were identified (Table 3). These gene products include known suppressor genes such as Smad1 and Mad1-like, as well as a number of genes with as yet poorly characterized roles in cancer.

TABLE 3
Genes located within a distance of 500 Kb of a methylated VIS

| tumor | | | | gene | protein |
|---|---|---|---|---|---|
| sample | gene | distance | human homologue | annotation | function |
| 99-16 | A kinase anchor | 44 kb 3′ | protein A kinase | NM_018747 | regulates PKA |
| band 1 | protein 7 | | anchor protein 7 | | distribution, |
| | | | isoform gamma | | probably to |
| | | | | | cytoplasm |
| | arginase 1, liver | 200 kb 5′ | arginase-1 | NM_007482 | liverenzyme, |
| | | | | | ureumcyclus |
| | cofactor required for | 210 kb 3′ | idem | NM_027347 | co-activator of |
| | Sp1 trancriptional | | | | transcription by |
| | activation subunit 3 | | | | Sp1 |
| | erytrocyte protein 4.1- | 280 kb 3′ | band 4.1 like protien 2 | | x |
| | like | | | | |
| | ectonucleotide | 300 kb 5′ | idem | NM_134005 | hydrolysis of |
| | pyrophosphatase/phosphodiesterase 3 | | | | extracellular |
| | | | | | nucleotides |
| | ectonucleotide | 400 kb 5′ | idem | NM_008813 | hydrolysis of |
| | pyrophosphatase/phosphodiesterase 1 | | | | extracellular |
| | | | | | nucleotides |
| 99-19 | cyclin D3 | intron 1 | G1/S-specific cyclin | NM_007632 | G1 to S-phase |
| band 1 | | | D3 | | transmission, |
| | | | | | phosphorylation |
| | | | | | of rb, |
| | taube nuss | 3.3 kb 5′ | idem | NM_022015 | required for |
| | | | | | basal and |
| | | | | | activator- |
| | | | | | dependent |
| | | | | | transcription, |
| | | | | | TATA-binding |
| | | | | | protein initiation |
| | | | | | factor |
| | unknown seq | 36 kb 5′ | x | x | x |
| | Riken cDNa | 68 kb 3′ | x | x | x |
| | 1700001C19 | | | | |
| | bystin | 92 kb 5′ | idem | | bystin is found in |
| | | | | | the placenta |
| | | | | | from the sixth-tenth |
| | | | | | week of |
| | | | | | pregnancy |
| | guanylate cyclase | 105 kb 5′ | Guanylyl cyclase | NM_008189 | retinal |
| | activator 1a (retina) | | activating protein 1 | | |
| | | | (GCAP 1) | | |
| | Trf (TATA binding | 110 kb 5′ | Ubiquitin specific | NM_020048 | de-ubiquitination |
| | protein-related factor)- | | protease homolog 49 | | |
| | proximal protein | | | | |
| | homolog (Drosophila) | | | | |
| | ubiquitin specific | 125 kb 5′ | Ubiquitin carboxyl- | NM_198421 | de-ubiquitination |
| | peptidase 49 | | terminal hydrolase 49 | | |
| | guanylate cyclase | 110 kb 3′ | Guanylyl cyclase | NM_146079 | retinal |
| | activator 1B | | activating protein 2 | | |
| | | | (GCAP 2) | | |
| | mitochondrial | 125 kb 3′ | Mitochondrial 28S | NM_183086 | x |
| | ribosomal protein S10 | | ribosomal protein S10 | | |
| | transcriptional | 150 kb 3′ | idem | NM_172622 | basal cell cycle |
| | regulating factor 1 | | | | regulatory |
| | | | | | protein |
| | | | | | interacting with |
| | | | | | Sp1 to activate |
| | | | | | the p21 and p27 |
| | | | | | gene promoters |
| | fibroblast growth factor | 190 kb 5′ | idem | NM_144939 | FRS3 negatively |
| | receptor substrate 3 | | | | regulates ERK2 |
| | | | | | signaling |
| | | | | | activated via |
| | | | | | EGF stimulation |
| | | | | | through direct |
| | | | | | binding to ERK2 |
| | progastricsin | 225 kb 5′ | Gastricsin precursor | NM_025973 | |
| | (pepsinogen C) | | | | |
| | transcription factor EB | 280 kb 5′ | idem | NM_011549 | TFE3 and TFEB |
| | | | | | regulate E- |
| | | | | | cadherin and |
| | | | | | WT1 expression |
| | forkhead box P4 | 375 kb 3′ | forkhead box protein | NM_028767 | members of the |
| | | | P4 | | forkhead box |
| | | | | | gene family, |
| | | | | | including |
| | | | | | members of |
| | | | | | subfamily P, |
| | | | | | have roles in |
| | | | | | mammalian |
| | | | | | oncogenesis |
| 99-36 | DNA primase, p58 | intron 7 | DNA primase large | NM_008922 | synthesizes |
| band 2 | subunit | | subunit | | small RNA |
| | | | | | primers for the |
| | | | | | Okazaki |
| | | | | | fragments made |
| | | | | | during |
| | | | | | discontinuous |
| | | | | | DNA replication |
| | RIKEN 1700001G17 | 95 kb 5′ | x | x | x |
| | gene | | | | |
| | Rab23, member of | 150 kb 5′ | RAS related protien | NM_008999 | GTPase |
| | RAS proto-oncogene | | Rab23 | | mediated signal |
| | family | | | | transduction and |
| | | | | | intracellular |
| | | | | | protein |
| | | | | | transportation |
| | Bcl2-associated | 175 kb 3′ | BAG-family molecular | NM_145392 | The BAG |
| | athanogene 2 | | chaperone regulator 2 | | domains of |
| | | | | | BAG1, BAG2, |
| | | | | | and BAG3 |
| | | | | | interact |
| | | | | | specifically with |
| | | | | | the Hsc70 |
| | | | | | ATPase domain |
| | | | | | in vitro and in |
| | | | | | mammalian |
| | | | | | cells. All 3 |
| | | | | | proteins bind |
| | | | | | with high affinity |
| | | | | | to the ATPase |
| | | | | | domain of Hsc70 |
| | | | | | and inhibit its |
| | | | | | chaperone |
| | | | | | activity in a Hip- |
| | | | | | repressible |
| | | | | | manner |
| | zinc finger protein 451 | 190 kb 3′ | zinc finger protein | NM_133817 | |
| | | | 451 | | |
| | dystonin | 340 kb 5′ | Bullous pemphigoid | NM_010081, | |
| | | | antigen 1 isoforms | NM_133833, | |
| | | | 1/2/3/4/5/8 | NM_134448 | |
| 99-36 | lunatic fringe gene | intron 1 | Beta-1,3-N- | NM_008494 | embryonic |
| band 4 | homolog | | acetylglucosaminyltransferase | | development |
| | | | lunatic | | |
| | | | fringe | | |
| | 12 days embryo | 5.5 kb 5′ | x | x | x |
| | eyeball cDNA, RIKEN | | | | |
| | full-length enriched | | | | |
| | library, | | | | |
| | clone: D230015O06 | | | | |
| | tweety homologue 3 | 10.5 kb 3′ | tweety 3 | NM_175274 | chloride channel |
| | | | | | activity |
| | galectin-related inter- | 45 kb 5′ | PREDICTED: similar | XM_132470 | |
| | fiber protein | | to galectin-related | XP_132470 | |
| | | | inter-fiber protein | | |
| | carbohydrate | 85 kb 3′ | idem | NM_021528 | carbohydrate |
| | sulfotransferase 12 | | | | metabolism |
| | IQ motif containing E | 55 kb 3′ | idem | NM_028833 | |
| | guanine nucleotide | 150 kb 3′ | Guanine nucleotide- | NM_010302 | Gα(12) |
| | binding protein, alpha | | binding protein, | | stimulates cell |
| | 12 | | alpha-12 subunit | | proliferation and |
| | | | | | neoplastic |
| | | | | | transformation of |
| | | | | | NIH 3T3 cells by |
| | | | | | attenuating |
| | | | | | p38MAPK- |
| | | | | | associated |
| | | | | | apoptotic |
| | | | | | responses, while |
| | | | | | activating the |
| | | | | | mitogenic |
| | | | | | responses |
| | | | | | through the |
| | | | | | stimulation of |
| | | | | | ERK- and JNK- |
| | | | | | mediated |
| | | | | | signaling |
| | | | | | pathways, |
| | | | | | results from |
| | | | | | differential |
| | | | | | proteome |
| | | | | | analysis report a |
| | | | | | role for SET in |
| | | | | | Gα(12)-mediated |
| | | | | | signaling |
| | | | | | pathways and a |
| | | | | | role for Gα(12) in |
| | | | | | the regulation of |
| | | | | | the leukemia- |
| | | | | | associated SET- |
| | | | | | protein |
| | | | | | expression |
| | caspase recruitment | 260 kb 3′ | Caspase recruitment | NM_175362 | genetic |
| | domain family, member | | domain protein 11 | | inactivation of |
| | 11 | | | | the MAGUK |
| | | | | | family protein |
| | | | | | CARD11/Carma |
| | | | | | 1/Bimp3 results |
| | | | | | in a complete |
| | | | | | block in T and B |
| | | | | | cell immunity. |
| | | | | | CARD11 is |
| | | | | | essential for |
| | | | | | antigen receptor- |
| | | | | | and PKC- |
| | | | | | mediated |
| | | | | | proliferation and |
| | | | | | cytokine |
| | | | | | production in T |
| | | | | | and B cells due |
| | | | | | to a selective |
| | | | | | defect in JNK |
| | | | | | and NFκB |
| | | | | | activation |
| | eukaryotic translation | 170 kb 3′ | Eukaryotic translation | NM_133916 | results indicate |
| | initiation factor 3, | | initiation factor 3 | | that p116 plays |
| | subunit 9 (eta) | | subunit 9 (elF-3 eta) | | an essential role |
| | | | | | in the early |
| | | | | | stages of mouse |
| | | | | | development |
| | sorting nexin 8 | 220 kb 5′ | idem | NM_172277 | |
| | FtsJ homolog 2 | 280 kb 5′ | Putative ribosomal | NM_013393, | FTSJ2 is a |
| | | | RNA | NM_177442 | nucleolar RNA |
| | | | methyltransferase 2 | | methyltransferase |
| | | | | | involved in |
| | | | | | eukaryotic RNA |
| | | | | | processing and |
| | | | | | modification |
| | nudix (nucleoside | 275 kb 3′ | 7,8-dihydro-8- | NM_008637 | MTH1 protects |
| | diphosphate linked | | oxoguanine | | cells from H2O2- |
| | moiety X)-type motif 1 | | triphosphatase | | induced cell |
| | | | | | dysfunction and |
| | | | | | death by |
| | | | | | hydrolyzing |
| | | | | | oxidized purine |
| | | | | | nucleotides |
| | | | | | including 8-oxo- |
| | | | | | dGTP and 2-OH- |
| | | | | | dATP |
| | mitotic arrest deficient | 290 kb 5′ | Mitotic spindle | NM_010752 | 1. MAD1 and |
| | 1-like 1 (Mad1-like) | | assembly checkpoint | | Proto-Oncogene |
| | | | protein MAD1 | | Proteins c-myc |
| | | | | | reciprocally |
| | | | | | regulate |
| | | | | | ribosomal DNA |
| | | | | | transcription, |
| | | | | | providing a |
| | | | | | mechanism for |
| | | | | | coordination of |
| | | | | | ribosome |
| | | | | | biogenesis and |
| | | | | | cell growth 2. |
| | | | | | Together these |
| | | | | | data |
| | | | | | demonstrate that |
| | | | | | the MYC- |
| | | | | | antagonist |
| | | | | | MAD1 and |
| | | | | | cyclin-dependent |
| | | | | | kinase inhibitor |
| | | | | | p27(Kip1) |
| | | | | | cooperate to |
| | | | | | regulate the self- |
| | | | | | renewal and |
| | | | | | differentiation of |
| | | | | | HSCs in a |
| | | | | | context- |
| | | | | | dependent |
| | | | | | manner. 3. Data |
| | | | | | show that the |
| | | | | | loss of Trrap |
| | | | | | leads to |
| | | | | | chromosome |
| | | | | | missegregation, |
| | | | | | mitotic exit |
| | | | | | failure and |
| | | | | | compromised |
| | | | | | mitotic |
| | | | | | checkpoints, |
| | | | | | which are |
| | | | | | caused by |
| | | | | | defective Trrap- |
| | | | | | mediated |
| | | | | | transcription of |
| | | | | | the mitotic |
| | | | | | checkpoint |
| | | | | | proteins Mad1 |
| | | | | | and Mad2. |
| 99-44 | Stearoyl-CoenzymeA | 95 kb 3′ | Acyl-Coa desaturase | NM_005063 | by globally |
| band 1 | desaturase 1 | | | | regulating lipid |
| | | | | | metabolism, |
| | | | | | stearoyl-CoA |
| | | | | | desaturase |
| | | | | | activity |
| | | | | | modulates cell |
| | | | | | proliferation and |
| | | | | | survival and |
| | | | | | shows the role of |
| | | | | | endogenously |
| | | | | | synthesized |
| | | | | | monounsaturated |
| | | | | | fatty acids in |
| | | | | | sustaining the |
| | | | | | neoplastic |
| | | | | | phenotype of |
| | | | | | transformed cells |
| | Stearoyl-CoenzymeA | intron 4 | Acyl-Coa desaturase | | |
| | desaturase 2 | | | | |
| | Stearoyl-CoenzymeA | 60 kb 3′ | Acyl-Coa desaturase | | |
| | desaturase 3 | | | | |
| | Stearoyl-CoenzymeA | 33 kb 5′ | Acyl-Coa desaturase | | |
| | desaturase 4 | | | | |
| | cDNA sequence | 110 kb 5′ | Polycystic kidney | NM_016112 | x |
| | BC046386 | | disease 2-like 1 | | |
| | | | protein | | |
| | biogenesis of | 150 kb 5′ | biogenesis of | XM_193940 | |
| | lysosome-related | | lysosome-related | | |
| | organelles complex-1, | | organelles complex- | | |
| | subunit 2 | | 1, subunit 2 isoform 1 | | |
| | CWF19-like 1, cell | 160 kb 5′ | idem | XM_129328 | |
| | cycle control (S. pombe) | | | XP_129328 | |
| | wingless related MMTV | 190 kb 5′ | Wnt-8b protein | NM_011720 | |
| | integration site 8b | | precursor | | |
| | gene model 341 | 220 kb 3′ | S. cerevisiae SEC31- | XM_140784 | WD40 domain |
| | | | like 2 isoform a | XP_140784 | |
| | NADH dehydrogenase | 250 kb 3′ | NADH-ubiquinone | NM_026061 | |
| | (ubiquinone) 1 beta | | oxidoreductase ASHI | | |
| | subcomplex 8 | | subunit, mitochondrial | | |
| | | | precursor | | |
| | hypoxia-inducible | 255 kb 5′ | Hypoxia-inducible | NM_176958 | |
| | factor 1, alpha subunit | | factor 1 alpha | | |
| | inhibitor | | inhibitor | | |
| | paired box gene 2 | 450 kb 5′ | Paired box protein | NM_011037 | |
| | | | Pax-2 | | |
| | conserved helix-loop- | 190 kb 5′ | Inhibitor of nuclear | NM_007700 | New nuclear role |
| | helix ubiquitous kinase | | factor kappa-B kinase | | of IKK-alpha in |
| | | | alpha subunit | | modifying |
| | | | | | histone function |
| | | | | | that is critical for |
| | | | | | the activation of |
| | | | | | NF-kappaB- |
| | | | | | directed gene |
| | | | | | expression |
| | SPFH domain family, | 215 kb 5 | SPFH domain protein | NM_145502 | |
| | member 1 | | 1 precursor | | |
| | cytochrome P450, | 260 kb 5′ | x | NM_001001446 | |
| | family 2, subfamily c, | | | | |
| | polypeptide 44 | | | | |
| | carboxypeptidase N, | 310 kb 5′ | Carboxypeptidase N | NM_030703 | carboxypeptidase |
| | polypeptide 1 | | catalytic chain | | N regulates the |
| | | | precursor | | biologic activity |
| | | | | | of SDF-1alpha |
| | | | | | by reducing the |
| | | | | | chemokine- |
| | | | | | specific activity |
| | dynamin binding | 390 kb 5′ | idem | NM_028029 | functions to bring |
| | protein | | | | together |
| | | | | | dynamin with |
| | | | | | actin regulatory |
| | | | | | proteins |
| | ATP-binding cassette, | 460 kb 3′ | Canalicular | NM_013806 | This protein is a |
| | sub-family C | | multispecific organic | | member of the |
| | (CFTR/MRP), member 2 | | anion transporter 1 | | MRP subfamily |
| | | | | | which is involved |
| | | | | | in multi-drug |
| | | | | | resistance, |
| | | | | | multispecific |
| | | | | | organic anion |
| | | | | | transporter |
| 99-48 | SMAD1 | exon 2 | idem | NM_008539 | Smad1 has a |
| band 1 | | | | | role in regulating |
| | | | | | p38 MAPK, |
| | | | | | Smad1, beta- |
| | | | | | catenin and Tcf4 |
| | | | | | have roles in |
| | | | | | controlling Myc |
| | | | | | transcription, |
| | | | | | Smad1 is an |
| | | | | | effector of |
| | | | | | signals provided |
| | | | | | by the bone |
| | | | | | morphogenetic |
| | | | | | protein (BMP) |
| | | | | | sub-group of |
| | | | | | TGFbeta |
| | | | | | molecules |
| | methylmalonic aciduria | 75 kb 5′ | Methylmalonic | NM_133823 | |
| | (cobalamin deficiency) | | aciduria type A | | |
| | type A | | protein, mitochondrial | | |
| | | | precursor | | |
| | PREDICTED: | 125 kb 5′ | x | x | x |
| | hypothetical protein | | | | |
| | LOC67687 | | | | |
| | OTU domain | 300 kb 5′ | Putative HIV-1- | XM_194424 | HIV-1 induced |
| | containing 4 | | induced protein HIN-1 | XP_194424 | protein HIN-1 |
| | ATP-binding cassette, | 300 kb 3′ | ATP-binding cassette | NM_015751 | Alternatively |
| | sub-family E (OABP), | | sub-family E member 1 | | referred to as the |
| | member 1 | | | | RNase L |
| | | | | | inhibitor, this |
| | | | | | protein functions |
| | | | | | to block the |
| | | | | | activity of |
| | | | | | ribonuclease L. |
| | | | | | Activation of |
| | | | | | ribonuclease L |
| | | | | | leads to |
| | | | | | inhibition of |
| | | | | | protein synthesis |
| | | | | | in the 2- |
| | | | | | 5A/RNase L |
| | | | | | system, the |
| | | | | | central pathway |
| | | | | | for viral |
| | | | | | interferon action |
| | anaphase promoting | 340 kb 5′ | idem | NM_026904 | |
| | complex subunit 10 | | | | |
| 99-56 | G-protein coupled | exon 3 | G-protein coupled | NM_173398 | ?? |
| band 3 | receptor 171 | | receptor H963 | | |
| | purinergic receptor | intron 1 | P2Y purinoceptor 14 | NM_001008497, | |
| | P2Y, G-protein | | (P2Y14) | NM_133200 | |
| | coupled, 14 | | | | |
| | mediator of RNA | intron 11 | x | XM_887994 | |
| | polymerase II | | | | |
| | transcription, subunit | | | | |
| | 12 homolog (yeast)-like | | | | |
| | G protein-coupled | 63 kb 3′ | Probable G protein- | NM_032399 | |
| | receptor 87 | | coupled receptor 87 | | |
| | Usher syndrome 3A | 225 kb 5′ | Usher syndrome type | NM_153384, | retinal and inner |
| | homolog (human) | | 3 protein | NM_153385, | ear |
| | | | | NP_700434, | malformations |
| | | | | NP_700435 | |
| | 15 days embryo head | 200 kb 3′ | immunoglobulin | | x |
| | cDNA, RIKEN full- | | superfamily, member | | |
| | length enriched library, | | 10 | | |
| | clone: 4022435C0 | | | | |
| | purinergic receptor | 190 kb 3′ | P2Y purinoceptor 13 | NM_028808 | |
| | P2Y, G-protein coupled | | | | |
| | 13 | | | | |
| | purinergic receptor | 195 kb 3′ | P2Y purinoceptor 12 | NM_027571 | |
| | P2Y, G-protein coupled | | | | |
| | 12 | | | | |
| | seven in absentia 2 | 430 kb 5′ | x | NM_009174 | Siah proteins |
| | | | | | function as E3 |
| | | | | | ubiquitin ligase |
| | | | | | enzymes to |
| | | | | | target the |
| | | | | | degradation of |
| | | | | | diverse protein |
| | | | | | substrates, an |
| | | | | | expansion of |
| | | | | | myeloid |
| | | | | | progenitor cells |
| | | | | | in the bone |
| | | | | | marrow of Siah2 |
| | | | | | mutant mice |
| 99-56 | WAS protein family, | intron 1 | Wiskott-Aldrich | NM_153423 | WAVE2 acts as |
| band 4 | member 2 | | syndrome protein | | the primary |
| | | | family member 2 | | effector |
| | | | | | downstream of |
| | | | | | Rac to achieve |
| | | | | | invasion and |
| | | | | | metastasis, |
| | | | | | suggesting that |
| | | | | | suppression of |
| | | | | | WAVE2 activity |
| | | | | | holds a promise |
| | | | | | for preventing |
| | | | | | cancer invasion |
| | | | | | and metastasis, |
| | | | | | WAVEs (WASP- |
| | | | | | family verprolin- |
| | | | | | homologous |
| | | | | | proteins) |
| | | | | | regulate the |
| | | | | | actin |
| | | | | | cytoskeleton |
| | | | | | through |
| | | | | | activation of |
| | | | | | Arp2/3 complex |
| | D164 sialomucin-like 2 | 50 kb 5′ | CD164 sialomucin- | XM_131719, | ulti- |
| | | | like 2 | XM_900155, | glycosylated |
| | | | | XM_900160 | core |
| | | | | | protein |
| | | | | | 24 |
| | | | | | (MGC- |
| | | | | | 24) |
| | mitogen-activated | 65 kb 5′ | idem | NM_016693 | The encoded |
| | protein kinase kinase | | | | kinase was |
| | kinase 6 | | | | identified by its |
| | | | | | interaction with |
| | | | | | MAP3K5/ASK, a |
| | | | | | protein kinase |
| | | | | | and an activator |
| | | | | | of c-Jun kinase |
| | | | | | (MAPK7/JNK) |
| | | | | | and |
| | | | | | MAPK14/p38 |
| | | | | | kinase, |
| | | | | | apoptosis signal- |
| | | | | | regulating kinase 2 |
| | AT hook, DNA binding | 90 kb 3′ | idem | NM_146155 | |
| | motif, containing 1 | | | | |
| | solute carrier family 9 | 200 kb 5′ | Sodium/hydrogen | NM_016981 | mice lacking |
| | (sodium/hydrogen | | exchanger 1 | | NHE1 |
| | exchanger), member 1 | | (Na(+)/H(+) | | upregulate their |
| | | | exchanger 1) | | Na(+) channel |
| | | | | | expression in the |
| | | | | | hippocampal and |
| | | | | | cortical regions |
| | | | | | selectively; this |
| | | | | | leads to an |
| | | | | | increase in Na(+) |
| | | | | | current density |
| | | | | | and membrane |
| | | | | | excitability |
| | Gardner-Rasheed | 175 kb 3′ | Proto-oncogene | NM_010208 | Hck and Fgr |
| | feline sarcoma viral | | tyrosine-protein | | function as |
| | (Fgr) oncogene | | kinase FGR | | negative |
| | homolog | | | | regulators of |
| | | | | | myeloid cell |
| | | | | | chemokine |
| | | | | | signaling by |
| | | | | | maintaining the |
| | | | | | tonic |
| | | | | | phosphorylation |
| | | | | | of PIR- |
| | G-protein coupled | 75 kb 3′ | Probable G-protein | NM_008154 | Gpr3-defective |
| | receptor 3 | | coupled receptor | | mice may |
| | | | GPR3 | | constitute a |
| | | | | | relevant model |
| | | | | | of premature |
| | | | | | ovarian failure |
| | | | | | due to early |
| | | | | | oocyte aging |
| | synaptotagmin-like 1 | 100 kb 3′ | synaptotagmin-like | NM_031393 | SHD of Slp1/Jfc1 |
| | | | protein 1 | | specifically and |
| | | | | | directly binds the |
| | | | | | GTP-bound form |
| | | | | | of Rab27A |
| | WD and | 150 kb 3′ | WD and | NM_199306 | WD40 domain |
| | tetratricopeptide | | tetratricopeptide | | |
| | repeats 1 | | repeats protein 1 | | |
| | nuclear distribution | 350 kb 3′ | Nuclear migration | NM_010948 | |
| | gene C homolog | | protein nudC | | |
| | (Aspergillus) | | | | |
| | nuclear receptor | 380 kb 5′ | Nuclear receptor 0B2 | NM_011850 | SHP acts as a |
| | subfamily 0, group B, | | (Orphan nuclear | | transcriptional |
| | member 2 | | receptor SHP) | | coregulator by |
| | | | | | inhibiting the |
| | | | | | activity of |
| | | | | | various nuclear |
| | | | | | receptors |
| | | | | | (downstream |
| | | | | | targets) via |
| | | | | | occupation of the |
| | | | | | coactivator- |
| | | | | | binding surface |
| | | | | | and active |
| | | | | | repression |
| | G patch domain | 400 kb 5′ | G patch domain | NM_172876 | |
| | containing 3 | | containing protein 3 | | |
| | ATP binding domain 1 | 410 kb 5′ | idem | | of the MDR/TAP |
| | family, member B | | | | subfamily are |
| | | | | | involved in |
| | | | | | multidrug |
| | | | | | resistance |
| | stratifin | 430 kb 3′ | 14-3-3 protein sigma | NM_018754 | Stratifin was first |
| | | | | | identified as an |
| | | | | | epithelial cell |
| | | | | | antigen |
| | | | | | exclusively |
| | | | | | expressed in |
| | | | | | epithelia. the |
| | | | | | functional role of |
| | | | | | sfn in cell |
| | | | | | proliferation and |
| | | | | | apoptosis could |
| | | | | | be relevant to |
| | | | | | the regulation of |
| | | | | | growth and |
| | | | | | differentiation as |
| | | | | | a tumor |
| | | | | | suppressor |
| | | | | | gene, stratifin |
| | | | | | itself is subject to |
| | | | | | regulation by |
| | | | | | p53 upon DNA |
| | | | | | damage and by |
| | | | | | epigenetic |
| | | | | | deregulation and |
| | | | | | Gene silencing |
| | | | | | of 14-3-3sigma |
| | | | | | by CpG |
| | | | | | methylation has |
| | | | | | been found in |
| | | | | | many human |
| | | | | | cancer types |
| | zinc finger, DHHC | 430 kb 3′ | x | NM_001017968 | |
| | domain containing 18 | | | | |
| | phosphatidylinositol | 480 kb 3′ | phosphatidylinositol | NM_178698 | |
| | glycan, class V | | glycan class V | | |
| | syntaxin 12 | 280 kb 5′ | idem | NM_133887 | |
| | protein phosphatase 1, | 325 kb 5′ | Nuclear inhibitor of | NM_146154 | NIPP1 has a role |
| | regulatory (inhibitor) | | protein phosphatase 1 | | in the nuclear |
| | subunit 8 | | | | targeting and/or |
| | | | | | retention of PP1 |
| | replication protein A2 | 400 kb 3′ | Replication protein A | NM_011284 | Phosphorylation |
| | | | 32 kDa subunit | | of the RPA2 |
| | | | | | subunit is |
| | | | | | observed after |
| | | | | | exposure of cells |
| | | | | | to ionizing |
| | | | | | radiation (IR) |
| | | | | | and other DNA- |
| | | | | | damaging |
| | | | | | agents, which |
| | | | | | implicates the |
| | | | | | modified protein |
| | | | | | in the regulation |
| | | | | | of DNA |
| | | | | | replication after |
| | | | | | DNA damage or |
| | | | | | in DNA rep |
| | sphingomyelin | 410 kb 5′ | Acid | NM_133888 | |
| | phosphodiesterase, | | sphingomyelinase- | | |
| | acid-like 3B | | like | | |
| | | | phosphodiesterase | | |
| | | | 3b precursor | | |
| | X Kell blood group | 440 kb 5′ | X Kell blood group | NM_201368 | |
| | precursor related family | | precursor-related | | |
| | member 8 homolog | | family, member 8 | | |
| | eyes absent 3 homolog | 450 kb 3′ | idem | NM_010166, | Experiments |
| | (Drosophila) | | | NM_210071, | performed in |
| | | | | NM_211356, | cultured |
| | | | | NM_211357 | Drosophila cells |
| | | | | | and in vitro |
| | | | | | indicate that |
| | | | | | Eyes absent has |
| | | | | | intrinsic protein |
| | | | | | tyrosine |
| | | | | | phosphatase |
| | | | | | activity and can |
| | | | | | autocatalytically |
| | | | | | dephosphorylate |
| | | | | | itself |
| 99-58 | cleavage stimulation | 20 kb 3′ | Cleavage stimulation | NM_024199 | is involved in the |
| band 1 | factor, 3′ pre-RNA, | | factor, 50 kDa subunit | | polyadenylation |
| | subunit 1 | | | | and 3′end |
| | | | | | cleavage of pre- |
| | | | | | mRNAs |
| | RIKEN cDNA | 23 kb 5′ | x | x | x |
| | F730031O20 gene | | | | |
| | aurora kinase A | 30 kb 3′ | Serine/threonine- | NM_011497 | serine/threonine |
| | | | protein kinase 6 | | mitotic kinase, |
| | | | | | BRCA1 |
| | | | | | phosphorylation |
| | | | | | by Aurora-A |
| | | | | | plays a role in |
| | | | | | G(2) to M |
| | | | | | transition of cell |
| | | | | | cycle, human |
| | | | | | cancer cells |
| | | | | | frequently exhibit |
| | | | | | overexpression |
| | | | | | of Aurora A |
| | | | | | protein |
| | | | | | regardless of the |
| | | | | | cell cycle stage |
| | RIKEN cDNA | 40 kb 5′ | x | x | x |
| | 2410001C21 gene | | | | |
| | (2410001C21Rik), | | | | |
| | mRNA | | | | |
| | RIKEN cDNA | 45 kb 5′ | x | x | x |
| | 2010011I20 gene | | | | |
| | (2010011I20Rik), | | | | |
| | mRNA | | | | |
| | Adult male spinal cord | 50 kb 3′ | OTTHUMP00000031 | x | x |
| | cDNA, RIKEN full- | | 350 (Fragment) | | |
| | length enriched library, | | | | |
| | clone: A330041C17 | | | | |
| | PREDICTED: | 70 kb 5′ | x | x | x |
| | hypothetical protein | | | | |
| | LOC76426 | | | | |
| | melanocortin 3 | 200 kb 3′ | idem | NM_008561 | |
| | receptor | | | | |
| | transcription factor AP- | 150 kb 5′ | Transcription factor | NM_009335 | AP-2 gamma |
| | 2, gamma | | Erf-1 | | seems to be |
| | | | | | required in early |
| | | | | | embryonic |
| | | | | | development, |
| | | | | | suggest a role of |
| | | | | | AP-2 |
| | | | | | transcription |
| | | | | | factors in the |
| | | | | | maintenance of |
| | | | | | a proliferative |
| | | | | | and |
| | | | | | undifferentiated |
| | | | | | state of cells, |
| | | | | | characteristics |
| | | | | | not only |
| | | | | | important during |
| | | | | | embryonic |
| | | | | | development but |
| | | | | | also in |
| | | | | | tumorigenesis |
| | cerebellin 4 precursor | 350 kb 5′ | cerebellin 4 precursor | NM_175631 | neuromodulatory |
| | protein | | | | function |
| | bone morphogenetic | 400 kb 3′ | bone morphogenetic | NM_007557 | BMP-7/OP-1, a |
| | protein 7 | | protein 7 precursor | | member of the |
| | | | | | transforming |
| | | | | | growth factor- |
| | | | | | beta (TGF-beta) |
| | | | | | family of |
| | | | | | secreted growth |
| | | | | | factors, is |
| | | | | | expressed |
| | | | | | during mouse |
| | | | | | embryogenesis |
| | | | | | in a pattern |
| | | | | | suggesting |
| | | | | | potential roles in |
| | | | | | a variety of |
| | | | | | inductive tissue |
| | | | | | interactions |
| 00-10 | myosin 1H | 40 kb 5′ | idem | NM_146163 | ?? |
| band 5 | | | | | |
| | forkhead box N4 | 40 kb 5′ | forkhead box protein | NM_148935 | expressed |
| | | | N4 | | during neural |
| | | | | | development in |
| | | | | | the retina, the |
| | | | | | ventral hindbrain |
| | | | | | and spinal cord |
| | | | | | and dorsal |
| | | | | | midbrain |
| | potassium channel | 45 kb 3′ | idem | NM_026145 | |
| | tetramerisation domain | | | | |
| | containing 10 | | | | |
| | acetyl-Coenzyme A | 65 kb 3′ | acetyl-Coenzyme A | NM_133904 | Acc2−/− mutant |
| | carboxylase beta | | carboxylase 2 | | mice have a |
| | | | | | normal life span, |
| | | | | | a higher fatty |
| | | | | | acid oxidation |
| | | | | | rate, and lower |
| | | | | | amounts of fat |
| | ubiquitin protein ligase | 83 kb 5′ | ubiquitin protein | NM_054093 | This gene |
| | E3B | | ligase E3 isoform B | | encodes a |
| | | | | | member of the |
| | | | | | E3 ubiquitin- |
| | | | | | conjugating |
| | | | | | enzyme family. |
| | | | | | The encoded |
| | | | | | protein may |
| | | | | | interact with |
| | | | | | other proteins |
| | | | | | and play a role in |
| | | | | | stress response. |
| | mevalonate kinase | 125 kb 5′ | idem | NM_023556 | Mevalonic |
| | | | | | aciduria, with |
| | | | | | psychomotor |
| | | | | | retardation, |
| | | | | | cerebellar ataxia, |
| | | | | | recurrent fever, |
| | | | | | and death in |
| | | | | | early childhood, |
| | | | | | and hyper- |
| | | | | | immunoglobulin |
| | | | | | D syndrome, |
| | | | | | with recurrent |
| | | | | | fever attacks |
| | | | | | without |
| | | | | | neurologic |
| | | | | | symptoms, are |
| | | | | | caused by |
| | | | | | mevalonate |
| | | | | | kinase deficiency |
| | methylmalonic aciduria | 120 kb 3′ | Cob(I)yrinic acid a,c- | NM_029956 | |
| | (cobalamin deficiency) | | diamide | | |
| | type B homolog | | adenosyltransferase, | | |
| | (human) | | mitochondrial | | |
| | | | precursor | | |
| | uracil DNA glycosylase | 175 kb 3′ | idem | NM_011677 | Immunoglobulin |
| | | | | | isotype switching |
| | | | | | is inhibited and |
| | | | | | somatic |
| | | | | | hypermutation |
| | | | | | perturbed in |
| | | | | | mice deficient in |
| | | | | | this enzyme |
| | ubiquitin specific | 200 kb 3′ | Ubiquitin carboxyl- | XM_149655 | |
| | peptidase 30 | | terminal hydrolase 30 | | |
| | transient receptor | 300 kb 3′ | idem | NM_022017 | Trpv4 gene in |
| | potential cation | | | | mice markedly |
| | channel, subfamily V, | | | | reduced the |
| | member 4 | | | | sensitivity of the |
| | | | | | tail to pressure |
| | | | | | and acidic |
| | | | | | nociception |
| | glycolipid transfer | 350 kb 3′ | idem | NM_019821 | |
| | protein | | | | |
| | G protein-coupled | 400 kb 3′ | G protein-coupled | NM_019834 | GIT proteins are |
| | receptor kinase- | | receptor kinase- | | GTPase- |
| | interactor 2 | | interactor 2 | | activating |
| | | | | | proteins (GAPs) |
| | | | | | for ADP- |
| | | | | | ribosylation |
| | | | | | factor (ARF) |
| | | | | | small GTP- |
| | | | | | binding proteins, |
| | | | | | and interact with |
| | | | | | the PIX family of |
| | | | | | Rac1/Cdc42 |
| | | | | | guanine |
| | | | | | nucleotide |
| | | | | | exchange |
| | | | | | factors. GIT and |
| | | | | | PIX transiently |
| | | | | | localize p21- |
| | | | | | activated protein |
| | | | | | kinases (PAKs) |
| | | | | | to remodeling |
| | | | | | focal adhesions |
| | | | | | through binding |
| | | | | | to paxillin |
| | ankyrin repeat domain | 460 kb 5′ | Ankyrin repeat | NM_026718 | |
| | 13a | | domain protein 13 | | |
| | D-amino acid oxidase 1 | 300 kb 3′ | idem | NM_010018 | |
| | slingshot homolog 1 | 325 kb 5′ | slingshot homolog 1 | NM_198109 | Expression of a |
| | (Drosophila) | | | | phosphatase- |
| | | | | | inactive SSH1 |
| | | | | | induces aberrant |
| | | | | | accumulation of |
| | | | | | F-actin and |
| | | | | | phospho-cofilin |
| | | | | | near the |
| | | | | | midbody in the |
| | | | | | final stage of |
| | | | | | cytokinesis and |
| | | | | | frequently leads |
| | | | | | to the regression |
| | | | | | of the cleavage |
| | | | | | furrow and the |
| | | | | | formation of |
| | | | | | multinucleate |
| | | | | | cells |
| | coronin, actin binding | 400 kb 5′ | Coronin-1C | NM_011779 | This gene |
| | protein 1C | | | | encodes a |
| | | | | | member of the |
| | | | | | WD repeat |
| | | | | | protein family. |
| | | | | | WD repeats are |
| | | | | | minimally |
| | | | | | conserved |
| | | | | | regions of |
| | | | | | approximately 40 |
| | | | | | amino acids |
| | | | | | typically |
| | | | | | bracketed by gly- |
| | | | | | his and trp-asp |
| | | | | | (GH-WD), which |
| | | | | | may facilitate |
| | | | | | formation of |
| | | | | | heterotrimeric or |
| | | | | | multiprotein |
| | | | | | complexes. |
| | | | | | Members of this |
| | | | | | family are |
| | | | | | involved in a |
| | | | | | variety of cellular |
| | | | | | processes, |
| | | | | | including cell |
| | | | | | cycle |
| | | | | | progression, |
| | | | | | signal |
| | | | | | transduction, |
| | | | | | apoptosis, and |
| | | | | | gene regulation, |
| | | | | | Coronin 3 is |
| | | | | | abundantly |
| | | | | | expressed in the |
| | | | | | adult CNS. All |
| | | | | | murine brain |
| | | | | | areas express |
| | | | | | coronin 3 during |
| | | | | | embryogenesis |
| | | | | | and the first |
| | | | | | postnatal stages |
| | selectin, platelet (p- | 480 kb 5′ | P-selectin | NM_009151 | The |
| | selectin) ligand | | glycoprotein ligand 1 | | homozygous |
| | | | precursor | | PSGL-1-deficient |
| | | | | | mouse was |
| | | | | | viable and fertile. |
| | | | | | The blood |
| | | | | | neutrophil count |
| | | | | | was modestly |
| | | | | | elevated, In |
| | | | | | contrast, |
| | | | | | leukocyte rolling |
| | | | | | 2 h after tumor |
| | | | | | necrosis factor |
| | | | | | alpha stimulation |
| | | | | | was only |
| | | | | | modestly |
| | | | | | reduced, but |
| | | | | | blocking |
| | | | | | antibodies to E- |
| | | | | | selectin infused |
| | | | | | into the PSGL-1- |
| | | | | | deficient mouse |
| | | | | | almost |
| | | | | | completely |
| | | | | | eliminated |
| | | | | | leukocyte rolling |
| 00-10 | hypothetical protein | 240 kb 5′ | x | XM_135684 | x |
| band 6 | LOC74236 | | | XP_135684 | |
| | expressed sequence | 200 kb 3′ | Melanoma-derived | x | x |
| | AI987692 | | leucine zipper- | | |
| | | | containing | | |
| | | | extranuclear factor | | |
| | RIKEN cDNA | 240 kb 3′ | Melanoma-derived | x | x |
| | 9930109F21 gene | | leucine zipper- | | |
| | (9930109F21Rik), | | containing | | |
| | mRNA | | extranuclear factor | | |
| | 0 day neonate thymus | 250 kb 3′ | Melanoma-derived | x | x |
| | cDNA, RIKEN full- | | leucine zipper- | | |
| | length enriched library, | | containing | | |
| | clone: A430110B17 | | extranuclear factor | | |
| | Protein FAM49B | 300 kb 3′ | Protein FAM49B (L1) | NM_016623 | |
| | | | | (homo | |
| | | | | sapiens) | |
| | development and | 500 kb 3′ | 130-kDa | NM_010026 | SH3 domain |
| | differentiation | | phosphatidylinositol | | |
| | enhancing | | 4,5-biphosphate- | | |
| | | | dependent ARF1 | | |
| | | | GTPase-activating | | |
| | | | protein | | |

1. Method for the identification of tumor suppressor genes comprising
a) infecting mice with a cancer causing retrovirus;
b) checking for the presence of methylated viral inserts; and
c) identifying the genes flanking the viral insertion site.

2. Method according to claim 1, wherein the genomic DNA is randomly cut to provide fragments containing the viral inserts.

3. Method according to claim 1 or 2, further comprising a enrichment of methylated DNA fragments, preferably by immunoprecipitating said methylated DNA fragments.

4. Method according to claim 3, wherein the immunoprecipation is performed with an antibody directed against 5-methyl-cytosine (α-5mC).

5. Method according to claim 1 or 2, wherein the methylated fragments are amplified, preferably by inverse PCR.

6. Tumor suppressor gene selected from the group consisting of A kinase anchor protein 7, arginase 1 from liver, cofactor required for Sp1 transcriptional activation subunit 3, erythrocyte protein 4.1-like, ectonucleotide pyrophosphatase/phosphodiesterase 3, ectonucleotide pyrophosphatase/phosphodiesterase 1, cyclin D3, taube nuss, Riken cDNa 1700001C19, bystin, guanylate cyclase activator 1a (retina), Trf (TATA binding protein-related factor)-proximal protein homolog, ubiquitin specific peptidase 49, guanylate cyclase activator 1B, mitochondrial ribosomal protein S10, transcriptional regulating factor 1, fibroblast growth factor receptor substrate 3, progastricsin (pepsinogen C), transcription factor EB, forkhead box P4, DNA primase, p58 subunit, RIKEN 1700001G17 gene, Rab23, Bc12-associated athanogene 2, zinc finger protein 451, dystonin, lunatic fringe gene homolog, 12 days embryo eyeball cDNA, RIKEN full-length enriched library, clone:D230015006, tweety homologue 3, galectin-related inter-fiber protein, carbohydrate sulfotransferase 12, IQ motif containing E, guanine nucleotide binding protein α2, caspase recruitment domain family member 11, eukaryotic translation initiation factor 3, subunit 9, sorting nexin 8, FtsJ homolog 2, nudix (nucleoside diphosphate linked moiety X)-type motif 1, Stearoyl-CoenzymeA desaturase 1, Stearoyl-CoenzymeA desaturase 2, Stearoyl-CoenzymeA desaturase 3, Stearoyl-CoenzymeA desaturase 4, cDNA sequence BC046386, biogenesis of lysosome-related organelles complex-1 subunit 2, CWF19-like 1 cell cycle control, wingless related MMTV integration site 8b, gene model 341, NADH dehydrogenase (ubiquinone) 1 beta subcomplex 8, hypoxia-inducible factor 1 α subunit inhibitor, paired box gene 2, conserved helix-loop-helix ubiquitous kinase, SPFH domain family member 1, cytochrome P450 family 2 subfamily c polypeptide 44, carboxypeptidase N polypeptide 1, dynamin binding protein, ATP-binding cassette sub-family C (CFTR/MRP) member 2, methylmalonic aciduria (cobalamin deficiency) type A, hypothetical protein LOC67687, OTU domain containing 4, ATP-binding cassette sub-family E (OABP) member 1, anaphase promoting complex subunit 10, G-protein coupled receptor 171, purinergic G-protein coupled receptor P2Y 14, purinergic G-protein coupled receptor P2Y 13, purinergic G-protein coupled receptor P2Y 12, mediator of RNA polymerase II transcription subunit 12 homolog (yeast)-like, G protein-coupled receptor 87, Usher syndrome 3A homolog, 15 days embryo head cDNA RIKEN full-length enriched library clone:4022435C0, seven in absentia 2, WAS protein family member 2, D164 sialomucin-like 2, mitogen-activated protein kinase kinase kinase 6, AT hook DNA binding motif containing 1, solute carrier family 9 (sodium/hydrogen exchanger) member 1, Gardner-Rasheed feline sarcoma viral (Fgr) oncogene homolog, G-protein coupled receptor 3, synaptotagmin-like 1, WD and tetratricopeptide repeats 1, nuclear distribution gene C homolog, nuclear receptor subfamily 0 group B member 2, G patch domain containing 3, ATP binding domain 1 family member B, stratifin, zinc finger DHHC domain containing 18, phosphatidylinositol glycan class V, syntaxin 12, protein phosphatase 1 regulatory (inhibitor) subunit 8, replication protein A2, acid-like sphingomyelin phosphodiesterase 3B, X Kell blood group precursor related family member 8 homolog, eyes absent 3 homolog (Drosophila), cleavage stimulation factor 3′ pre-RNA, subunit 1, RIKEN cDNA F730031020 gene, aurora kinase A, RIKEN cDNA 2410001C21 gene (2410001C21Rik) mRNA, RIKEN cDNA 201001I20 gene (2010011I20Rik) mRNA, Adult male spinal cord cDNA RIKEN full-length enriched library clone:A330041C17, hypothetical protein LOC76426, melanocortin 3 receptor, transcription factor AP-2 gamma, cerebellin 4 precursor protein, bone morphogenetic protein 7, myosin 1H, forkhead box N4, potassium channel tetramerisation domain containing 10, acetyl-Coenzyme A carboxylase beta, ubiquitin protein ligase E3B, mevalonate kinase, methylmalonic aciduria (cobalamin deficiency) type B homolog (human), uracil DNA glycosylase, ubiquitin specific peptidase 30, transient receptor potential cation channel subfamily V member 4, glycolipid transfer protein, G protein-coupled receptor kinase-interactor 2, ankyrin repeat domain 13a, D-amino acid oxidase 1, slingshot homolog 1 (Drosophila), coronin actin binding protein IC, selectin platelet (p-selectin) ligand, hypothetical protein LOC74236, expressed sequence A1987692, RIKEN cDNA 9930109F21 gene (9930109F21Rik) mRNA, 0 day neonate thymus cDNA RIKEN full-length enriched library clone:A430110B17, Protein FAM49B development and differentiation enhancing.

7. Use of a tumor suppressor gene from Table 3 for diagnosis of AML, more preferably, wherein said diagnosis comprises classification of AML subtypes and/or determination of susceptibility to therapy.

8. Use of a tumor suppressor gene from Table 3 for therapy of AML.

9. Method for therapy of AML by increasing the expression and/or availability of a tumor suppression gene of table 3.

10. Method according to claim 3, wherein the methylated fragments are amplified, preferably by inverse PCR.

11. Method according to claim 4, wherein the methylated fragments are amplified, preferably by inverse PCR.
